(19) United States
(12) Patent Application Publication
(10) Pub. No.: US 2012/0296261 A1
(43) Pub. Date: Nov. 22, 2012

(54) DELIVERY DEVICE AND DELIVERY METHODS

(76) Inventors: David Jonathan Townley Whitaker, London (GB); Craig Harvey Nelson, Herts (GB); David Morrison Russell, Cambridge (GB); Thomas Mark Kemp, Ashwell (GB); Simon Francis Brereton, Cambridge (GB); Philip Andrew Dent, Cambridge (GB); Paul Farenden, Hertforshire (GB)

(21) Appl. No.: 13/510,117

(22) PCT Filed: Nov. 17, 2010
(86) PCT No.: PCT/GB2010/002116

§ 371 Date: Jul. 31, 2012

(30) Foreign Application Priority Data
Nov. 17, 2009 (GB) 0920125.2

(51) Int. Cl. A61F 9/00; A61M 37/00

(52) U.S. Cl. 604/20; 604/294

(57) ABSTRACT

Various delivery devices and methods are disclosed, including a delivery device (200) for delivering a dose substance (110) in powdered form to an eye. The device can include a pocket (100A, 100B, 100C) for holding dose substance and a propulsion system (202, 204, 908, 1004, 1006) for propelling a dose consisting of a predetermined volume of said dose substance from said pocket towards said eye, wherein one or both of said pocket and said propulsion system are configured to inhibit propulsion of particles having a size greater than a predetermined threshold to said eye.

This is a U.S. National Phase Application under 35 U.S.C. §371 of PCT/GB2010/002116, filed Nov. 17, 2010, which claims the benefit of priority under 35 U.S.C. §119 to GB Application Number 0920125.2, filed Nov. 17, 2009, both of which are incorporated herein by reference in their entireties.

The present invention relates to devices and methods for delivering doses of a substance to an eye, and in particular for delivering doses of a substance in powdered form. The delivered substance may be a medicament for treatment of the eye, or for systemic treatment via the eye, for example. The substance may include a vaccine, a disclosing agent (dye), vitamins, or be configured for cosmetic or recreational use (e.g. cooling eye drops).

Various devices are available in the prior art for providing doses of ocular fluid.

For example, dropper bottles containing a large number of doses of an ocular fluid are in widespread use. A dose from such a device is typically applied by positioning the bottle above the eye and squeezing or otherwise manipulating the bottle to cause a single drop to fall from the opening of the bottle into the eye. This operation can be facilitated by controlling the surface tension of the fluid and the material and/or size and shape of the bottle opening. Nevertheless, the action can be difficult to carry out, particularly for fragile users, as it requires sustained elevation of the bottle, a significant degree of force and control, and the user to tilt their head back or lie down.

In practice, the size of drops dispensed by dropper bottles varies substantially, which can lead to uncertainty about the dose being applied to the patient. More generally, dropper bottles tend to be effective only for relatively large drops, i.e. drops containing more fluid than can actually be sustained on the eye. For example, dropper bottles may dispense drops of between 50 μl and 100 μl, whereas the eye can only sustain about 20 μl. This results in unpleasant “run-off” during application, waste of the product being dispensed, and variation in the dose.

Blow-fill-sealed single ampoules can be used as an alternative to the dropper bottle. As the ampoules are intended for single use and are only opened for the first time immediately before use, sterility is reliably maintained without any added preservatives. However, the method of application is very similar to that of the dropper bottle and the same disadvantages are encountered. Furthermore, ampoules have a twist-off portion which must be removed to access the dose, the process of which can create irregularities at the dropper tip which can further reduce the accuracy of droplet size and form a dangerous or uncomfortable surface for bringing into contact with or close to the eye. In particular, the delivery action is difficult to carry out and the drop size tends to be larger than the eye can sustain, leading to unpleasant run-off and wastage.

It is an object of the present invention to at least partially overcome some of the problems with the prior art mentioned above.

According to an aspect of the invention, there is provided a delivery device for delivering a dose substance in powdered form to an eye, comprising: a pocket for holding dose substance; and a propulsion system for propelling a dose consisting of a predetermined volume of said dose substance from said pocket towards said eye; wherein one or both of said pocket and said propulsion system are configured to inhibit propulsion of particles having a size greater than a predetermined threshold to said eye.

In comparison with systems relying on liquid drops or sprays, the above arrangement facilitates more accurate dose delivery and/or lower losses of dose substance. The provision of a propulsion system means the device is easier to use than systems which rely on gravity; it is not orientation sensitive and there is no need to tilt the head back or hold the device above the head. Inhibiting propulsion of particles that are too large (above the predetermined threshold) further improves comfort of use, by avoiding irritation of, and/or damage to, the eye. Run-off due to excess dose substance is avoided because no additional liquid is delivered. In addition, the viscosity on the eye surface can remain high, which helps to increase residency time of the substance on the eye.

Compared to liquid drops or sprays, powdered substances also tend to have a longer shelf life. The use of preservatives can thus be avoided or reduced. Powdered substances tend also to be more stable, and formulations can be achieved which are not practical in liquid drops or sprays. In addition, it is possible to control spatial distribution of the spray to a greater degree than is possible with liquid drops or sprays.

The device may be configured such that a majority (or substantially all) of the dose substance present in the pocket is propelled towards the eye each time the device is actuated. In other words, the pocket may be designed to hold only one dose at a time. Alternatively, the pocket may be configured to hold a plurality of doses, with the propulsion system and/or pocket being arranged such that only a relatively small proportion of the dose substance available in the pocket is propelled out of the pocket each time the device is actuated. In this case, it is the nature of the propulsion system and/or pocket rather than the amount of dose substance that is initially present in the pocket that ensures that only a predetermined volume of the dose substance is propelled towards the eye. Alternatively or additionally, the device may comprise a plurality of pockets, each being configured to hold a single dose or a plurality of doses. In this case, means may be provided for displacing the pockets so that unused pockets can be brought sequentially from storage positions to a position at which the dose or doses therein can be propelled towards the eye. When the pockets have been used, they may be moved to a position at which they can be removed from the device and disposed of. Alternatively or additionally, means may be provided for storing the used pockets within the device for disposal at a later time, possibly along with the device itself.

The pocket may be arranged to hold the dose substance in a thin layer. The thin layer may be such that the thickness of the layer at any given point is less than 10 percent of the lateral width (i.e. of the smallest lateral dimension) of the layer. Preferably, the layer is even thinner than this, for example less than 1 percent or less than 0.1 percent. The thin layer may even comprise a single layer of particles, such that the thickness of the layer at any given point is substantially equal to the diameter of the particles present at that point. This approach reduces the total contact area between individual grains of the powder and helps to avoid clumping together of the grains to form larger particles. This may improve comfort (because of the reduction in average particle size) and dose accuracy (because fewer particles are too large to be propelled to the eye or otherwise filtered out). Additionally, the lack of head space in the pocket facilitates accurate filling of the pocket via volumetric means.

The propulsion system may comprise a gas source for providing a flow of gas to said pocket. Optionally, the pocket may comprise a surface with which said dose substance will be in contact in use, and the propulsion system may be configured to direct said flow of gas towards said surface from the side of said surface where the dose is in contact, or over said surface on the side of said surface where the dose is in contact. This approach reduces interference with the gas stream between the gas source and the dose to be expelled from the pocket, thus facilitating control of the properties of the gas stream (e.g. rate of flow, direction, turbulence) when it reaches the dose, relative to alternative arrangements in which the gas stream must pass through filters or membranes before reaching the dose substance.

Preferably, the inhibition of propulsion of particles having a size greater than a predetermined threshold to the eye is achieved without introducing any membrane-like or collision-based filters between the pocket and the eye. In other words, it is preferable that no provision is made for selectively removing larger particles by way of a mechanism that relies on them being more likely to collide with an obstacle than smaller particles (e.g. a membrane with holes that are smaller than the size of the particles to be filtered). Instead, embodiments of the present invention include ways of controlling the nature of the propulsion system and/or the pocket to achieve the control of particle size. Alternatively or additionally, the shape of the channels leading from the pocket to the eye may be controlled to favour smaller particles. These general approaches increase the proportion of the dose that reaches the eye and help to improve dose accuracy and/or minimize waste.

The delivery device may comprise a gas source and a flow controller, with the gas controller being configured to control a rate of flow of gas over the pocket such that only particles smaller than a predetermined size will reach the eye. Larger particles are either not expelled from the pocket or fall short of the eye in their trajectory between the pocket and the eye. This constitutes an effective and easily adaptable methodology for filtering by particle size.

The term “gas source” is intended to cover any means by which a flow of gas is produced, including any means for compressing air or any other gas, or releasing pre-compressed gas, in order to create the flow.

The pocket may comprise a flat surface for supporting the dose substance, and the propulsion system may be configured to propel the dose substance towards the eye by directing a flow of gas onto or over the flat surface, for example at an oblique angle thereto or substantially parallel thereto. This approach tends to produce a particle stream or flux that has a horizontally elongated cross-section, which corresponds more closely to the shape of the eye. The cross-section can also be varied according to the size of the eye, for example by controlling the size of the flat surface, the distribution of dose substance thereon, and the flow rate and/or direction of the gas flow onto or over the flat surface. By “flat”, what is meant is substantially flat or sufficiently flat for the horizontally elongated cross-section to be achieved to a significant extent.

Alternatively, the flow could be arranged to be through or partially through the pocket. In other words, the pocket could be designed so as to constrain the flow laterally in more than one lateral direction within the pocket (rather than just in one lateral direction, as with a flow impinging on a flat surface), for example in substantially all lateral directions (e.g. the pocket may be tubular and the gas flow axial).

According to an alternative aspect of the invention, there is provided a delivery device for delivering a dose substance in powdered form to an eye, comprising: a pocket for holding dose substance; and a propulsion system for propelling a dose consisting of a predetermined volume of said dose substance from said pocket towards said eye; said propulsion system comprising: a primary gas source for providing a primary flow of gas that flows over or through said pocket and is effective to propel said dose towards said eye; and a secondary gas source for providing a secondary flow of gas that is separate from said primary flow of gas and which is effective for increasing the proportion of said dose that impinges on said eye.

The primary gas source may comprise a bellows, which provides gas when compressed. Alternatively or additionally, the primary gas source may comprise means for directing gas provided by a user, for example via blowing into a tube, releasing gas from a container of compressed gas, or via manual compression of a piston.

Examples of secondary flows are as follows.

A secondary flow can be provided to induce interception between different flows in the vicinity of the eye to reduce the velocity of gas flow near the eye and help increase the proportion of dose substance that reaches the eye and stays in contact with the eye.

Alternatively or additionally, a secondary flow can be provided in the form of an “air curtain” which surrounds the target eye before and alternatively or additionally during the delivery of the dose substance. This could help to control the local environment, prevent ingress of matter from the external environment, and/or prevent loss of drug.

Alternatively or additionally, a secondary flow could be provided onto the eye just prior to the primary air flow to induce a blink and therefore reduce the probability of blinking during delivery of the dose. Additionally, such an air flow might be used to modify the surface of the eye by changing chemical/adhesion properties and thus residency time of the delivered substance on the eye.

Alternatively or additionally, the secondary flow may be configured to disperse the fluid on the eye (rather than evaporate it) prior to delivery of the drug, so that the fluid on the eye pulls back under surface tension after delivery to thereby cover the drug beneath the fluid.

According to a further aspect of the invention, there is provided a delivery device for delivering a dose substance in powdered form to an eye, comprising: a pocket for holding dose substance; a propulsion system for propelling a dose consisting of a predetermined volume of said dose substance from said pocket towards said eye; and a mask configured in use to enclose a volume delimited by said mask and a portion of a face including said eye and through which volume said dose is to be propelled from said pocket to said eye.

Using a mask in this way enables greater control of the environment in the vicinity of the eye. The velocity of gas currents, gas composition and humidity can be controlled so as to maximize the proportion of dose substance delivered, adsorbed and absorbed by the eye (i.e. dose substance which enters the eye system in some way). The mask can also be configured to fit against the face in such a way as to aim the dose substance towards the eye in a repeatable manner, with a minimum of conscious effort from the user. Sensors (for example, pressure sensors, or other contact sensors) can be incorporated into the mask to detect when the mask is correctly in position and the system configured automatically to trigger delivery of the dose substance (when the mask is in place) or to block delivery of the dose substance (when the mask is not yet in place) as a function of the output of the sensors (which could be electronic or mechanical, for example). Blinking sensors may be provided for detecting blinking and the system may be adapted so as to initiate delivery of the dose substance at a timing that minimizes the chances of coinciding with a blinking event. Vents may be provided to avoid uncomfortable pressure differences between the inside of the mask and the external environment. Another benefit of preventing blinking is that it prevents more fluid from being added to the eye.

According to an alternative aspect of the invention, there is provided a method for delivering a dose substance in powdered form to an eye, comprising: holding dose substance in a pocket; propelling a dose consisting of a predetermined volume of said dose substance from said pocket towards said eye; and inhibiting delivery of particles having a size greater than a predetermined threshold.

According to an alternative aspect of the invention, there is provided a method for delivering a dose substance in powdered form to an eye, comprising: holding dose substance in a pocket; pressing a mask against a portion of a face at least partially surrounding said eye in order to define a volume delimited partially by said mask and partially by said face; propelling a dose consisting of a predetermined volume of said dose substance from said pocket through said volume to said eye.

The invention will be more clearly understood from the following description, given by way of example only, with reference to the accompanying drawings, in which:

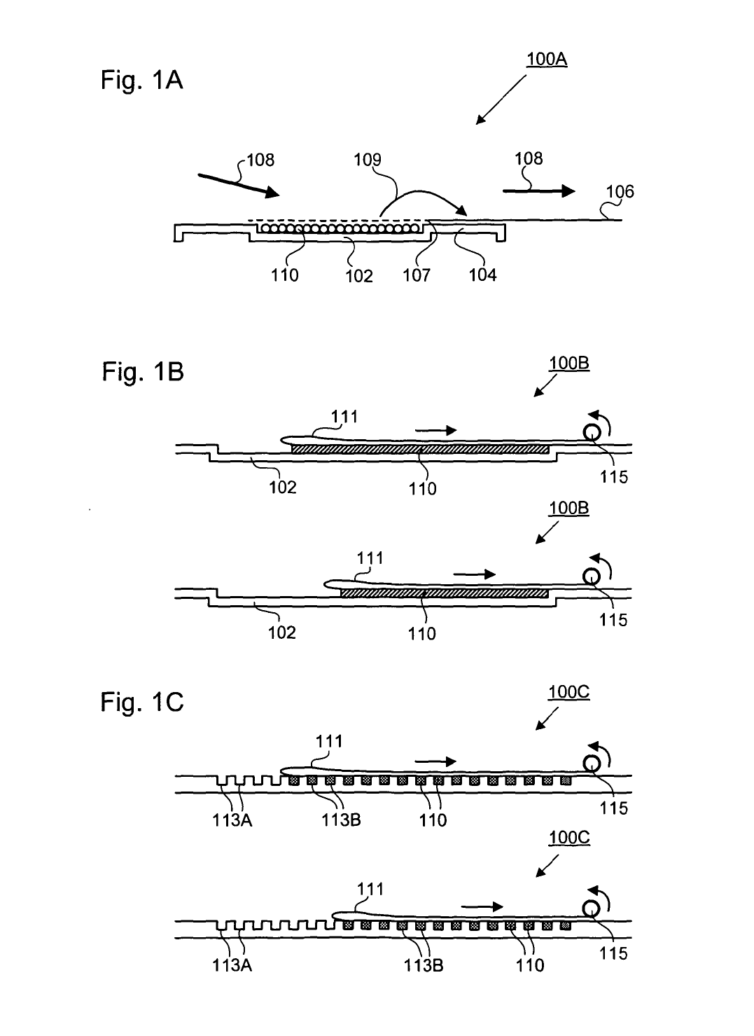

FIG. 1A is a schematic side view of a pocket for holding particles of dose substance in a thin layer, with a lid configured to open in a downstream direction;

FIG. 1B is a schematic side view of pocket according to an alternative embodiment, in which dose substance can be progressively exposed by peeling back a flexible lid, in order to vary the dose to be propelled;

FIG. 1C is a schematic side view of a pocket similar to that of FIG. 1B except that the pocket is divided into a plurality of discrete chambers;

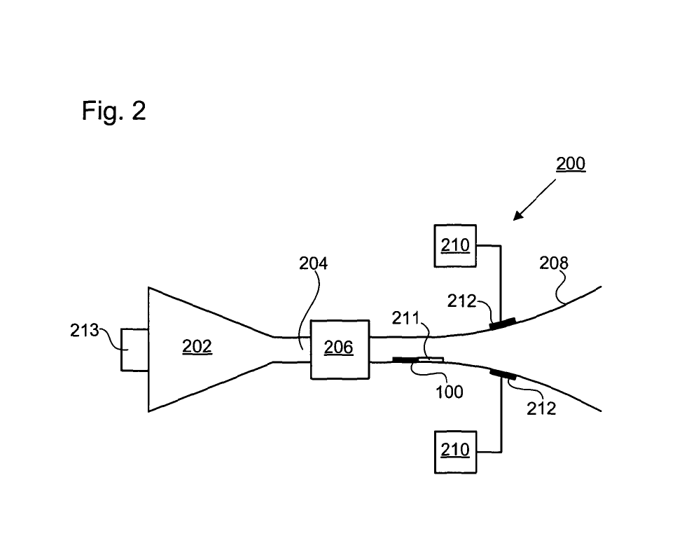

FIG. 2 is a schematic depiction of a propulsion system with various means for controlling how a dose of dose substance is propelled towards an eye, including charging devices, pressure valves and a divergent nozzle;

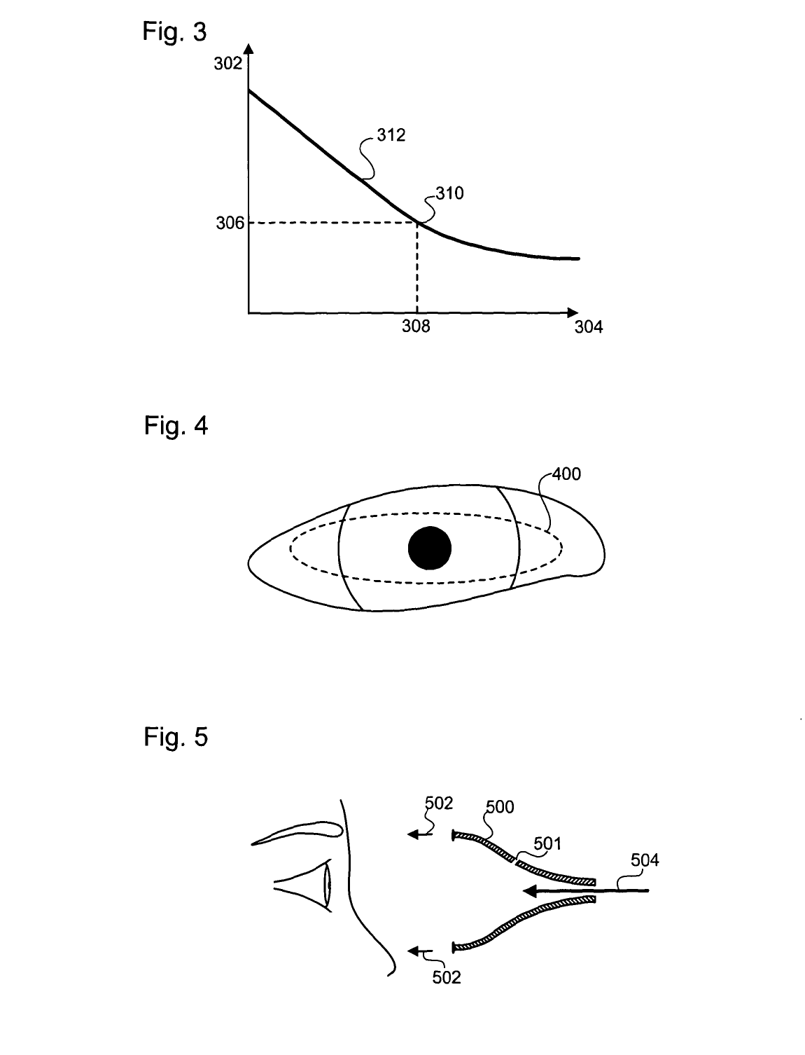

FIG. 3 is a graph showing how pressure in a gas canister decreases with the number of dispensed doses to illustrate operation of pressure-based control of particle size distributions using a pressure valve;

FIG. 4 shows a horizontally elongated target region on an eye;

FIG. 5 illustrates use of a mask;

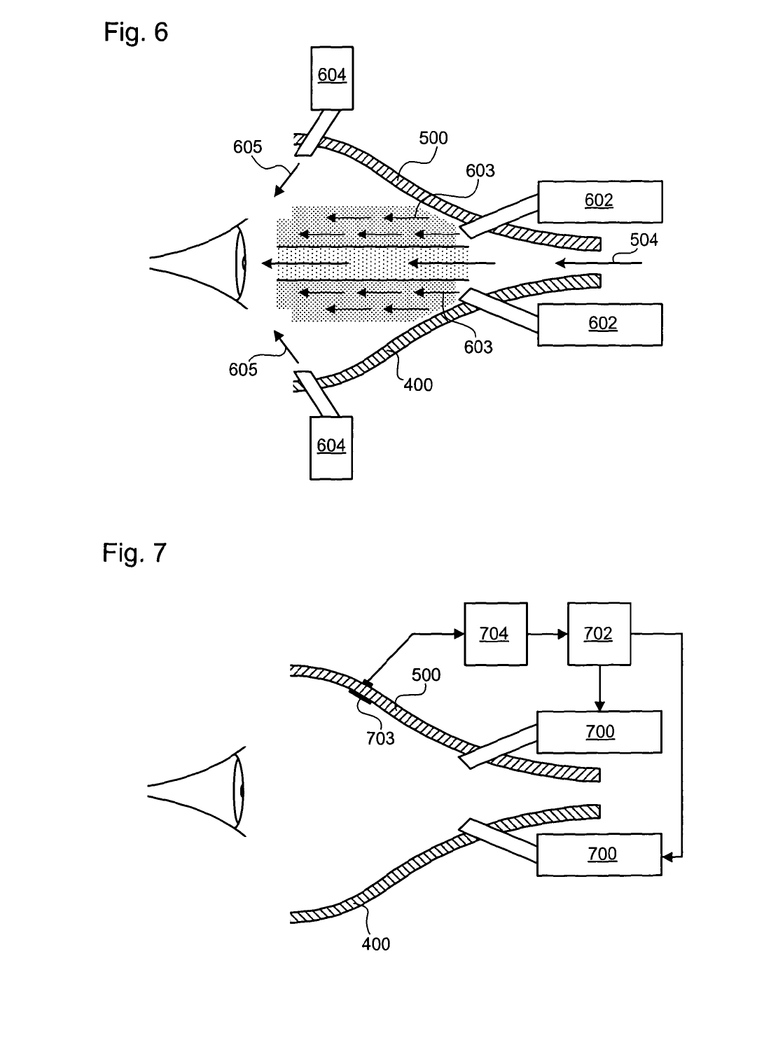

FIG. 6 is a schematic illustration of examples of a mask for use with gas sources for providing secondary flows of gas for laterally constraining a primary flow of gas and/or for increasing gas turbulence in the vicinity of the eye;

FIG. 7 is a schematic illustration of a mask having a gas source usable as either or both of a pre- and post-dose gas source for controlling the humidity of the eye respectively prior to and after propulsion of a dose of dose substance onto the eye;

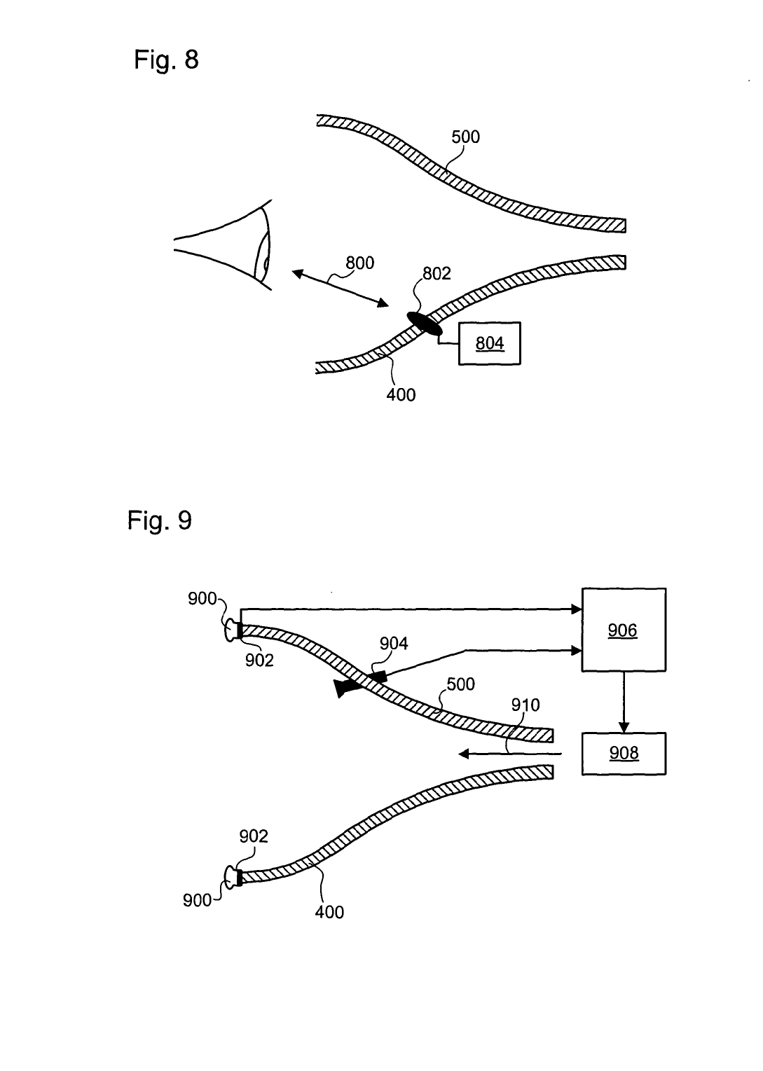

FIG. 8 is a schematic illustration of a mask having a focus target for helping a user to orient his eye in a way which makes it easier to direct dose substance onto a portion of the eye for which dose substance uptake is enhanced;

FIG. 9 is a schematic illustration of a mask having a blinking detector and pressure sensors that are configured to interact with a propulsion actuation controller;

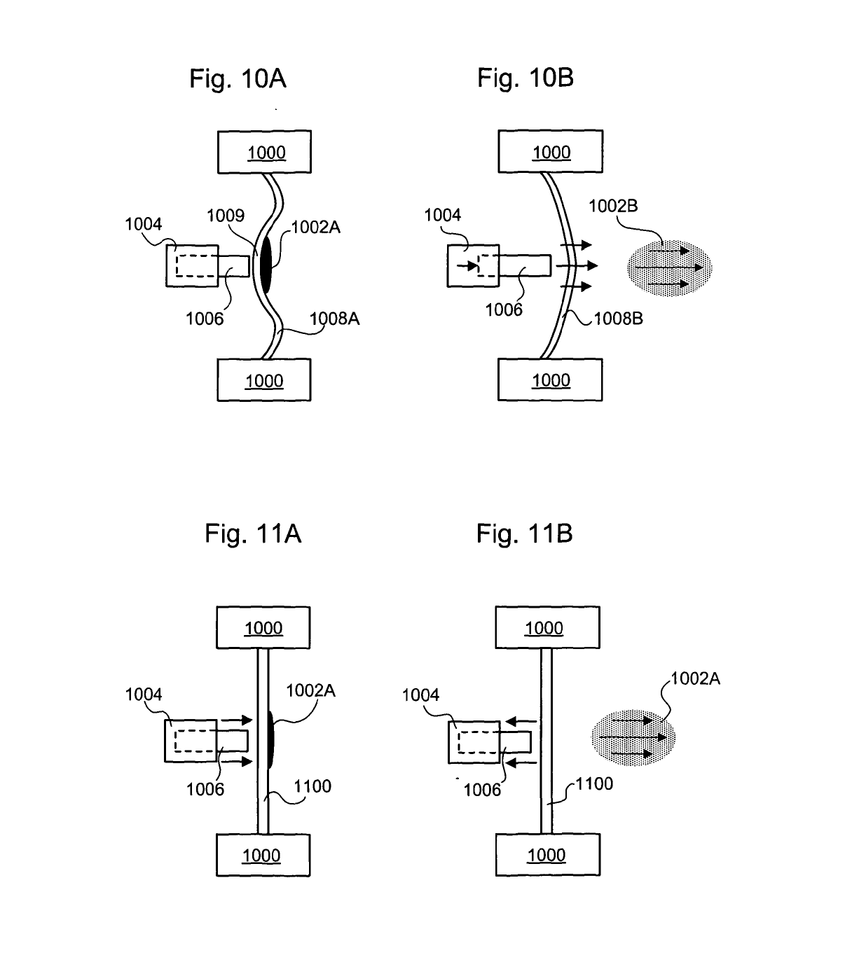

FIGS. 10A and 10B are schematic illustrations of a mechanically bi-stable element for use in an embodiment of the propulsion system;

FIGS. 11A and 11B are schematic illustrations of a flexible membrane and force imparting device for use in an embodiment of the propulsion system; and

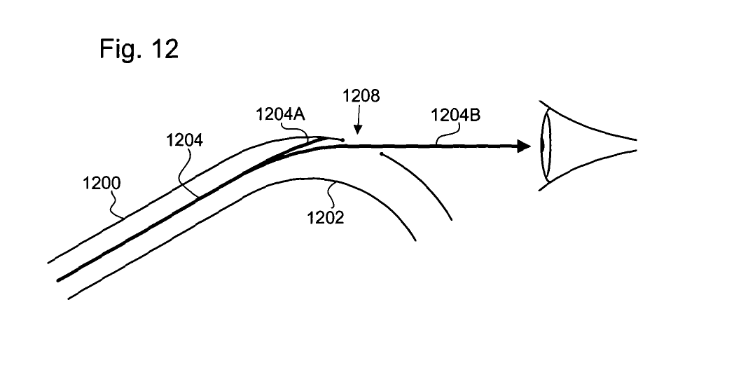

FIG. 12 is a schematic illustration of an arrangement for filtering dose substance particles according to size, so as only to allow particles that are within a pre-determined range to reach the eye.

FIG. 1A shows an example of a pocket 100A for holding an amount of dose substance 110 for eventual propulsion towards an eye using a propulsion system as described below. The dose substance 110 is provided in powdered form. Typically, the amount of dose substance contained in the pocket 100A corresponds to a single dose that is intended to be applied to the eye.

The dose substance may be a medicament for treatment of the eye, or for systemic treatment via the eye, for example. The substance may include a vaccine, a disclosing agent (dye), vitamins, or be configured for cosmetic or recreational use (e.g. cooling eye drops).

The powdered dose substance 110 may be held in the pocket in dry form. The pocket 100A may be configured to facilitate an industrial filling process, thereby helping to ensure accurate dosage and reduced costs.

In the case where substantially all of the dose substance 110 in the pocket 100A is expected to reach the eye and be absorbed thereby, the amount of dose substance 110 in the pocket 100A will be equal to the intended dose. This arrangement minimises waste of the dose substance. Alternatively, the system may be arranged so that only a portion (i.e. less than all) of the dose substance 110 held in the pocket 100A is expected to leave the pocket 100 and/or only a portion of the dose substance that does leave the pocket 100A is expected to reach the eye. In this case, the amount of dose substance provided in the pocket 100A will be greater than the desired dose, the amount of excess being chosen such that the portion expected to reach the eye is equal to the intended dose. Calibration measurements may be carried out in order to determine the amount of dose substance 110 that needs to be present at the pocket 100A in order to achieve a given intended dose, for example.

The pocket may alternatively be arranged to hold a quantity of dose substance that is equivalent to a plurality of individual doses and the propulsion system may be configured in this case to propel only a small proportion (corresponding to a single dose) of the total dose substance available each time the device is actuated.

Specific example embodiments having this functionality are described below with reference to FIGS. 1B and 1C.

The size of the particles reaching the eye should be sufficiently small to minimize or completely avoid physical irritation or damage to the eye. Small particle sizes are also more easily absorbed by the body and are thus advantageous from this point of view also.

According to embodiments of the invention, the pocket and/or propulsion system are arranged to control the size of particles that reach the eye and in particular to inhibit propulsion of particles having a size greater than a predetermined threshold. The predetermined threshold may correspond, for example, to the particle size at which irritation is first discernible by a user of the device. Preferably, the predetermined threshold may be around 10 microns in diameter. Alternatively, the predetermined threshold may be selected to be higher than this, to correspond for example to a size where discomfort for the user is clearly discernible, but at an acceptable level, to allow use of larger particles. For a given level of repeatability, the aim in general will be to control the particle size and shape distribution so that discomfort to the user is minimized, but not necessarily avoided entirely. A controlled amount of discomfort can be useful for providing feedback to the user, for example indicating to the user that a dose has successfully reached the eye.

Providing a powder in a sufficiently fine grade in said pocket may not be sufficient to ensure that the particles reaching the eye are below the predetermined threshold because, in the absence of countermeasures, individual grains of the powder held by the pocket will tend to clump together to form effective particles consisting of a plurality of individual grains.

The pocket 100A of FIG. 1A is arranged to reduce the extent to which individual grains of dose substance 110 clump or stick together by reducing contact between grains. The pocket 100A comprises a shallow indentation 102 formed within a substantially flat outer body 104. The indentation 102 is sufficiently wide and long to allow an amount of dose substance associated with at least a single dose to be spread out over the bottom of the indentation 102 in a thin layer, e.g. a single layer where the thickness of the layer at any given point is substantially equal to the diameter of the individual grains of the powder at that point. By reducing the extent to which (or even avoiding the situation where) individual grains can be in contact with other grains in all three dimensions (by favouring lateral contact between grains, within the plane of the thin or single layer), the extent to which grains can stick together within the pocket and form particles consisting of multiple grains is reduced.

The lateral size of the thin layer of substance can be sufficiently compact to be expelled efficiently, even when the layer is extremely thin or even a single layer, because the drug (or active ingredient) can be supplied in relatively pure form, meaning that the total volume of powder can be kept sufficiently low.

Alternatively, the pocket may be designed to be deeper and the flow configured so as to expel the dose substance from the pocket by means of the Venturi effect.

According to one embodiment, the pocket 100A is designed so that the dose substance 110 will be expelled from the pocket 100A by a flow of gas directed (arrows 108) over the pocket (incident at an oblique angle to the pocket or parallel to the pocket). The pocket 100A comprises a lid 106 which covers and protects the powder within the indentation 102 when closed (see broken line). In this embodiment, the lid 106 opens by pivoting about axis 107 in the direction shown by arrow 109, downstream relative to the flow of gas 108 which will be used to propel the dose substance 110 towards the eye. This arrangement ensures that the gas propelling the dose substance 110 flows over a surface of the lid 106 that was previously in contact with the dose substance when the lid 106 was closed. Any dose substance 110 that became attached to this inner surface of the lid 106 will thus tend to be entrained with the rest of the dose substance 110, thereby reducing loss of dose substance and helping to ensure accuracy of dose. In this particular example, the lid opens downstream, but any other opening direction (for example, upstream) could also achieve similar advantages if the flow is such as to be directed over the inner surface of the lid during use, with the effect of clearing or partially removing dose substance adhered to the lid section.

As mentioned above, the pocket and propulsion system may be arranged so that not all of the dose substance present in the pocket is expelled in a single actuation of the device. FIGS. 1B and 1C illustrate example arrangements for implementing this functionality.

In the embodiment shown in FIG. 1B, the pocket 100B is configured so that the dose substance 110 is contained within an indentation 102 similar to that in the embodiment of FIG. 1A. However, instead of having a lid 106 (flexible or rigid) that is switchable between a closed position that covers all of the dose substance 110 present in the indentation 102 and an open position in which all of the dose substance 110 is exposed, the lid 111 of this embodiment can be peeled back (or otherwise removed) gradually in incremental steps to expose less than all of the dose substance 110 present in the pocket 100B. The lid 111 may be flexible or have multiple hinges, for example. In this way, a single pocket can be used to provide dose substance for a plurality of separate deliveries. Alternatively or additionally, the size of individual doses can be varied controllably by changing the extent to which the lid 111 is peeled back between successive actuations. Optionally, the pocket 100B may be discarded after a single use, along with any remaining unexposed dose substance (beneath the portion of the lid 111 that did not get peeled back). The lower diagram in FIG. 1B represents the situation where the lid 111 has been peeled back relative to the configuration of the upper diagram in FIG. 1B by a distance which corresponds to a single dose, and the device has been actuated to expel the dose substance that was exposed by the movement/deformation of the lid 111.

FIG. 1C shows an embodiment with a flexible lid 111 similar to that of the embodiment of FIG. 1B. However, instead of having a continuous indentation 102 for containing the dose substance 110, a plurality of discrete indentations 113A/113B are provided, the indentations 113B underneath the lid 111 being completely full of dose substance and the indentations 113A outside of the lid 111 being completely empty of dose substance (due to a previous actuation of the device while these indentations 113A were exposed). As with the embodiment of FIG. 1B, this arrangement allows the amount of dose substance per dose to be varied controllably. The provision of discrete indentations helps to ensure that the dose is metered accurately and may serve as a visual aid for a user where the lid 111 is to be peeled back manually between doses. For example, a normal dose may correspond to the dose substance contained within five discrete indentations 113A/B, in which case the user would peel back the flexible lid 111 so as to expose five indentations prior to actuation of the device. The dose for a child may correspond to the dose substance contained within three discrete indentations 113/113B, for example, in which case the user would peel back the lid 111 to reveal just three indentations prior to actuation of the device. The lower diagram in FIG. 1C represents the situation where the lid 111 has been peeled back relative to the configuration of the upper diagram in FIG. 1C by a distance which corresponds to a single dose for a child, thus exposing the dose substance in three discrete indentations, and the device has been actuated to expel the dose substance that was exposed by the movement of the lid 111.

In the embodiments of FIGS. 1B and 1C, the lid 111 may be peeled back using a spool 115, for example, which may be driven electrically or manually.

The lid 106/111 of the pockets 100A/B/C may be impervious to the dose substance, so that none of the dose substance can penetrate through the lid 106/111 when expelled from the pocket 100A/B/C. Alternatively, the lid may be provided with holes which only allow particles of dose substance smaller than a threshold size to pass through (blocking larger particles), the lid thus serving as a particle-size filter. In this case, the lid may be configured to open up in use so as to be substantially perpendicular to the flow of dose substance between the pocket and the eye, so that only dose substance that passes through the lid can reach the eye.

Alternatively or additionally, there may be provided a separate particle filter (i.e. a particle filter that is not associated with the lid of the pocket), which is arranged so as to be in the flow path of the dose substance between the pocket and the eye. Alternatively or additionally, the lid may comprise two parts: a first part which is completely impervious to the dose substance and which is removed prior to actuation (without being placed so as to interrupt the flow of dose substance), and a second part which acts as a filter.

The particle-size filter may be configured to have an anti-bacterial action in addition to the particle size filtering action. Alternatively or additionally, a separate anti-bacterial filter may be provided.

FIG. 2 illustrates an example delivery device 200, for use with pockets 100A/B/C of the type shown in FIGS. 1A/B/C for example. In the embodiment shown in FIG. 2, further measures are provided to minimise the possibility of particles greater than the predetermined threshold reaching the eye.

According to this embodiment, a propulsion system for propelling a dose consisting of a predetermined volume of dose substance 110 from the pocket 100 to the eye consists of a bellows 202 and a channel 204. A gas input valve 213 is provided for allowing air to enter the bellows during expansion of the bellows. Preferably, the gas input valve 213 comprises an antibacterial filter to at least partially sterilize the air as it enters the bellows, thereby improving the quality of the air that is eventually blown onto the eye.

Actuation of the bellows 202 forces gas to travel through the channel 204 and over or through the pocket 100. Dose substance 110 present in the pocket 100 is entrained in the gas flow and propelled towards the eye.

As an alternative arrangement, a pressurized or liquefied gas canister and appropriate actuator could be provided instead of the bellows to provide the flow of gas through the channel 204.

A valve 206, for example a flap valve, serving as a “flow controller”, may be provided to ensure that the flow rate over the pocket 100 remains constant.

For example, in the case where a pressurized gas canister is used instead of the bellows 202, the pressure in the canister will fall during its lifetime. The functionality of the valve 206 in this case is described by reference to the schematic graph shown in FIG. 3. Here, the vertical axis 302 represents pressure and the horizontal axis 304 represents the number of doses that have been dispensed by the delivery device. The curve 312 represents schematically how the pressure within the pressurized gas canister declines during use. The valve 206 is effective to maintain the pressure within the channel at a constant target pressure 306 as long as the pressure in the pressurized gas canister remains above the target pressure 306. The pressure difference (and flow rate) across the region of the channel 204 containing the pocket 100 is thus kept constant up until the point 308 where the number of doses delivered by the device is such that the pressure within the canister declines below the target pressure 306 (crossover point 310). An alternative approach for achieving a constant pressure is to use a liquefied gas source.

For a given composition of particle and for most shapes of particle, smaller particles will follow a different trajectory when compared to larger particles, and particles over a certain size may not even leave the pocket 100. Careful selection of the target pressure 306 may therefore be used to prevent particles that are above a certain size (predetermined threshold) from reaching the eye. Calibration measurements may be used to determine the relationship between target pressure and the maximum size of particle that would reach the eye.

The average speed of gas particles (also referred to as “flow rate”) over/through the pocket may be varied by changing the cross-section of the channel in the vicinity of the pocket. Generally, a narrowing in the channel in which the pocket is positioned will lead to an increase in the average speed of gas particles over the pocket. Thus, the channel may be designed to be narrower in the vicinity of the pocket, so as to ensure a relatively high speed of gas flow over the pocket, which will help to expel the dose substance efficiently from the pocket. Additionally or alternatively, the surfaces in the region of the pocket may be configured to induce (or increase the level of) turbulence of the flow over or through the pocket, which would also generally increase the proportion of the dose substance that is expelled from the pocket for a given average gas flow speed. Downstream of the pocket (in between the pocket and the eye), the channel may be wider so as to reduce the speed of flow. Optionally, means may be provided for controllably changing the cross-section of the channel in the vicinity of the pocket to vary the speed and/or degree of turbulence of the gas flow and thereby the proportion of the dose substance that is expelled from the pocket and/or the cross-sectional shape of the stream of expelled dose substance.

Means may be provided for inducing a charge on particles for all or part of their trajectory towards the eye and/or for modifying the electric field in the region between the pocket 100 and the eye such that the proportion of dose substance that reaches the eye is increased, the average size of particles that reaches the eye is decreased, and/or the distribution of dose substance on the eye is improved (for example, spread out more uniformly or localized more effectively in a desired target region of the eye, for example an upper, outer region of the eye).

For example, as shown in FIG. 2, the delivery device 200 may comprise a charging device 211 for applying a charge to particles before they are propelled towards the eye. This may be achieved using the triboelectric effect, for example. For example, the pocket 100 may be formed from a material that will tend to charge the particles as they leave (i.e. as they transition between a state in which they are in direct contact with the material and a state in which they are no longer in direct contact with the material). The polarity and strength of the charging will vary according to the materials involved (e.g. the material of the portion of the pocket 100 or charging device 211 with which the particles come into contact and the material of the particles themselves), the surface roughness, surface energy, temperature, humidity and structural strains.

All of the particles charged in this way will have the same polarity of charge and so will tend to be repelled from each other. The average separation between particles will thus tend to increase and the rate of agglomeration (i.e. clumping together of individual powder grains to form particles that are larger than the individual grains) will decrease, any already agglomerated particles tending also to split up in flight into smaller particles. The overall effect will be a decrease in the average size of the particles that reach the eye.

The charging device 211 may also be configured to induce a potential difference between the particle and the eye, thus causing the dose substance to be attracted to the eye. This arrangement tends to increase the proportion of the dose substance 110 that reaches the eye, thus reducing loss and enhancing dose accuracy. The device could be configured such that there is a potential difference between the device and the user. This would maintain a common ground between the two. Electrodes may also be provided for connection to the human or animal to be treated in order to enhance electrostatic attraction of the dose substance particles to the eye and/or to help ensure that the degree of electrostatic attraction is repeatable/predictable. For example, the system may be configured to earth the user and the device while applying a charge to the particles.

The device 200 may also comprise electrodes 212 and electrical power sources 210, which can be used to modify the electrical field in the region through which the charged particles will be propelled, between the pocket 100 and the eye. This may be used to modify the cross-sectional profile of the particle flux. For example, arranging for the electrical field to point radially inwards towards the axis of the particle stream will tend to focus the particle stream (where the particles are positively charged) and, conversely, arranging for the electrical field to point radially outwards will tend to de-focus the particle stream (where the particles are positively charged). More complex electrical fields could be defined if desired to make more subtle changes to the particle stream cross-section. For example, the electrical field could be arranged so as to cause the particle stream cross-section to have a horizontally elongated form.

FIG. 4 shows an example of a shape of impact area (dotted line 400) that might result from a particle stream having a horizontally elongated form. As discussed above, this may be generated electrostatically. Alternatively, the shape of the pocket 100 and/or lid 106 and the way gas is blown over the pocket 100 by the propulsion system may be such as to generate the same effect. For example, the particles may be arranged so as to be spread out on a substantially planar portion of the pocket 100 in such a way that gas entrainment will naturally cause a horizontally elongated particle stream cross-section. The particular shape of the cross-section can be varied by changing the shape of the pocket 100 and/or lid 106 surface(s) and the way the powder is spread out over the surface, as well as changing parameters of the gas flow (e.g. rate of flow, angle of incidence onto the powder in the pocket, etc.).

More generally, manipulation of the particle stream cross-section may be used to spread the region of impact on the eye over as great an area as possible whilst minimizing contact (and therefore probable loss) with areas outside of the eye. For example, the horizontally elongated form may be made to correspond closely to the shape of the eye. For example, the horizontally elongated form may be substantially elliptical.

The flux of particles may even be manipulated in three dimensions so as to conform more closely to the three dimensional shape of the eye. For example, in addition to modifying the cross-section of the flux so as to be elongated in alignment with the eye, the profile of the flux may be modified so that the particles towards the center of the stream lag the particles towards the lateral periphery, in accordance with the convex curvature of the eye, so that all of the particles impact onto the eye at approximately the same time.

Even where the particles are not pre-charged using a charging device, an applied electric field may still be effective to exert some degree of control over the cross-section of the particle stream and on the average separation of particles within the stream.

The embodiment of FIG. 2 is also provided with a diverging nozzle 208, which is effective for reducing the rate of flow of gas towards the eye, while having little or no disadvantageous effect on the velocity of particles entrained by the gas flow towards the eye. Thus, according to this arrangement, for a given impact velocity of dose substance particles, the rate of flow of gas felt at the eye is reduced. This may improve the comfort of use and/or reduce the chances of dose substance being blown off target and/or lost due to excessive turbulence in the region of the eye.

Alternatively, the diverging nozzle 208 may be configured to reduce the velocity of the particles also, so as to reduce discomfort caused by particle impact. This arrangement allows the flow rate over or through the pocket to be increased (so as to improve the efficiency of particle entrainment) while at the same time minimizing discomfort to the user.

Although the bellows 202 and valve 206 are provided in the same embodiment as the charging device 211 and electrodes 212, and in the same embodiment as the diverging nozzle 208, these three sets of features may be provided separately or in other combinations, each being capable of providing advantages in the absence of the others.

The delivery device 200 may comprise a mask 500 which is to be brought into contact with the face in use so as substantially to enclose a volume delimited partly by a portion of the face and partly by the mask. FIG. 5 shows such an arrangement schematically with a portion of the mask 500 depicted in section. Arrows 502 represent movement of the mask onto the surface of the face ready for use and arrow 504 represents the direction in which dose substance will be propelled once the mask 500 is positioned against the face. By enclosing a volume of air around the eye prior to propulsion of the dose substance 110 towards the eye, unpredictable air currents originating from the external environment, which could otherwise cause dose substance to be blown off course and thereby lost, can be greatly inhibited, thus reducing losses and improving dose accuracy. In addition, the mask allows the distance between the pocket and the eye to be controlled.

The mask 500 may contain vents 501 to prevent build up of uncomfortable pressure differences between the inside of the mask 500 and the environment outside of the mask 500.

FIG. 6 depicts a delivery device comprising a mask 500 and secondary gas sources 602 and 604 for providing secondary gas flows that are separate from the gas flow that is used to carry the powdered dose substance towards the eye (arrows 504). Secondary gas sources 602 are configured to provide a secondary gas flow that is effective to constrain the flow of powdered dose substance laterally. For example, the secondary gas flow associated with the secondary gas sources 602 may surround the flow of powdered dose substance in all directions perpendicular to the flow direction of the dose substance. For example, the cross sectional profile of the secondary gas flow associated with the secondary gas sources 602 may be substantially annular. This situation is shown schematically in section in FIG. 6—see arrows 603.

Alternatively or additionally, the delivery device may comprise a secondary gas source 604 which is configured to direct a flow of gas (see arrows 605) towards a region in the vicinity of the eye in such a manner as to create interference between the gas flows near said eye. The interference created by this secondary flow of gas is effective to interact with the flow of gas carrying the dose substance and thereby reduce a net flow of gas onto the eye, without significantly reducing the velocity of particles of dose substance towards the eye. The risk of discomfort or irritation is thereby reduced and dose substance uptake can be enhanced. For example, where the dose substance particles are electrostatically attracted to the eye, the probability of this electrostatic attraction being overwhelmed by stray gas flows is reduced where turbulence is used to reduce an average flow rate of gas in the region of the eye.

FIG. 7 illustrates an alternative embodiment of the delivery device in which a mask 500 is provided with a mask humidity controller 704 for controlling the humidity of air within the mask 500. The mask humidity controller 704 may receive input from a gas humidity sensor 703 located within the mask 500 and respond to this input by controlling gas sources 700 and humidity source 702. For example the mask humidity controller 704 may be configured to adjust a flow rate of the gas sources 700 and/or a degree of humidity of gas output from the gas sources 700 in response to readings from the gas humidity sensor 703. Objective of humidity control is to optimise viscosity of fluid on the surface of the eye to improve the delivery of drug to the surface of the eye and then through the eye. The extent to which this will be possible and/or useful will vary according to the particular drug that is being delivered.

The mask humidity controller 704 of FIG. 7 may be arranged to reduce the humidity of the air in the vicinity of the eye in order to reduce the quantity of fluid on the surface of the eye, prior to propulsion of the powdered dose substance from the pocket 100. Reducing the quantity of fluid on the surface of the eye will tend to increase the residency time on the eye and thereby enhance absorption of the dose substance. This could be achieved by reducing the level of humidity in all of the volume enclosed by the mask 500 prior to delivery of the powdered dose substance. Methods of controlling moisture include: applying pre-dried gas, for example via a canister, and using a desiccant.

Providing a flow of gas onto the eye after the powdered dose substance has been delivered to the eye may be effective also to capture and redirect dose substance that has bounced off the eye back onto the eye. This mechanism is likely to be particularly effective where the flow of gas has a high humidity, especially when this takes the form of droplets of water entrained in the flow of gas. The breath of a user, which is relatively warm and humid and therefore comforting, may be used as a source for the flow of gas of high moisture content. Gas flows that are provided prior to delivery of the powdered dose substance may be referred to as pre-dose gas flows and the associated gas sources 700 as pre-dose gas sources 700. Gas flows provided after the powdered dose substance has been delivered may be referred to as post-dose gas flows and the associated gas sources 700 may be referred to as post-dose gas sources 700.

Alternatively or additionally, the pre-dose gas source 700 may be configured to interact with a surfactant reservoir (not shown) so as to deliver surfactant to the surface of the eye prior to delivery of the dose substance, in order to modify the surface tension of the eye in such a way as to improve uptake of the dose substance by the eye.

Alternatively or additionally, the pre-dose gas source 700 may be configured to direct a gas flow towards said eye just before delivery of the dose substance in order to induce blinking just before delivery and thereby reduce the probability of blinking during delivery. The timing of such a gas flow (i.e. the period of time between the gas flow and the dose substance delivery) should preferably be chosen so as to minimize the probability of blinking during delivery, by reference for example to experimental tests carried out at a variety of timings.

It is generally preferable to maximise the time that the dose substance spends on the surface of the eye in order to maximise the proportion of the dose substance that is absorbed by the body. One way in which this can be achieved is by increasing the viscosity of the fluid on the surface of the eye as described above, which prolongs the process of fluid draining. Another approach is to direct the flow of dose substance onto regions of the eye that are as far away as possible from the point towards which fluid drains, which is positioned towards the inner lower part of the eye. By “inner”, what is meant is towards the nose. This can be achieved partly by arranging for the mask 500 to be shaped such that when it is brought into contact with the face it fits in a location which is such that the flow of powdered dose substance will be directed towards an upper outer part of the eye. Alternatively or additionally, a user may be encouraged to look downwardly and inwardly (towards his nose) while the powder hits the eye so that subsequent movement of the eye towards a more central position will tend to transport the powder towards an upper outer region of the eye. In this latter arrangement, precise aiming of the powder becomes less important, which might enable manufacturing tolerances (or required accuracy of positioning by a user and/or ergonomic requirements) of the mask 500 and/or propulsion system to be relaxed relative to arrangements that rely on precise aiming of the particle stream into the upper outer portion of the socket, as well as making the device generally easier to use.

FIG. 8 is a schematic illustration of an embodiment comprising a focus target 802 for encouraging a user to direct his line of sight 800 in a downward and inward direction prior to propulsion of the dose substance onto the eye. The focus target 802 may be provided with a system of lenses to enable a user to focus on the focus target 802 (which will typically be located too close to the eye for it to be focused on without such assistance, particularly for older age patient groups). Additionally or alternatively, the focus target 802 may be driven by a power supply 804 so as to emit light and more effectively catch the attention of the user. Alternatively, a hole may be provided to allow light from the external environment to enter the mask, the outline of the hole defining the focus target. The power supply 804 (or a cover for the hole) might be linked to an actuation controller so that the focus target 802 only lights up when a user needs to focus on it (e.g. just prior to and during delivery of the dose substance). Extinction of the light might indicate that the dose has been delivered. The provision of a light also helps to reduce the probability of blinking during delivery of the dose.

The delivery device may comprise elements to control blinking (for example, to inhibit blinking temporarily) and/or elements to control the way the propulsion system operates in order to reduce the degree to which blinking may interfere with effective delivery of dose substance to the eye.

For example, a blinking detection device 904 may be provided. The blinking detection device 904 may comprise a CCD camera and suitable software for analysing the readings from the CCD camera, for example. Alternatively, a single light sensor could be used. Output from the blinking detection device 904 is fed to a propulsion actuation controller 906, which controls operation of the propulsion system 908 so as to reduce the probability of dose substance being propelled towards the eye at the same time as a blinking event occurs (such that dose substance ends up hitting the eye lid rather than the eye surface). Generally, the propulsion actuation controller 906 will attempt to control the timing at which the propulsion system 908 propels the dose substance so as to maximise the chances of it reaching the eye while the eye is open. For example, the propulsion actuation controller 906 may be configured to trigger propulsion of dose substance shortly after detection of a blinking event has been recorded by the blinking detection device 904, this period being associated with a relatively low probability of blinking. Alternatively or additionally, the blinking detection device 904 may be calibrated so as to be capable of detecting when a blinking event is about to occur (by monitoring the blink pattern) and the propulsion actuation controller 906 may be arranged to block operation of the propulsion system 908 in these circumstances.

The blinking detection device 904 may also be configured to record (via the CCD camera for example) when delivery has failed due to a blinking event happening at the wrong time.

Alternatively or additionally, leading edges of the mask 500 may be provided with cushions 900 and pressure sensors 902, as shown in FIG. 9. The pressure sensors 902 measure a contact pressure between the cushions 900 and the area around the eye socket with which the mask 500 has been brought into contact. In this embodiment, the mask 500 may be formed so as to fit against the face in such a way that when a pressure above a predetermined threshold is applied, blinking is substantially inhibited (for example, a user will find it substantially more difficult to blink than would be the case if the mask were not pressed against his face and/or the presence of the mask is such as to prevent involuntary blinking for the period while the user is using the delivery device and is aware that he should not blink). Output from the pressure sensors 902 is fed to the propulsion actuation controller 906 which controls the propulsion system 908 so that dose substance can only be propelled towards the eye (arrow 910) when the pressure measured by the pressure sensors 902 is equal to or above a predetermined threshold associated with the mask 500 being suitably pressed against the correct region of the face. A plurality of pressure sensors 902 may be provided that can independently measure the contact pressure at different points around the leading edge of the mask 500. In this way, more information is provided about how well the mask 500 is pressed against the face. For example, the provision of such a multiplicity of pressure sensors 902 would make it possible to detect when the mask 500 is not pressed against the correct portion of the face, this showing up generally as a non-uniform distribution of pressure around the leading edge of the mask 500. The propulsion actuation controller 906 may be further configured to prevent propulsion of the dose substance by the propulsion system 908 unless a suitable set of pressure measurements is received from the plurality of pressure sensors 902 (for example, a suitable set of pressure measurements may be where all of the pressure sensors 902 measure a contact pressure above the predetermined threshold and/or that all of the measured contact pressures are within a certain allowed range of each other).

The pressure sensors can operate electronically or mechanically, for example.

The provision of pressure sensors 902 and the associated propulsion actuation controller 906 have been discussed above in the context of a mask 500 adapted to fit against the face in such a way as to inhibit blinking. However, this configuration will also be useful even in the case where the mask 500 is not configured to inhibit blinking. Where a mask 500 is to be used, it is generally the case that propulsion of the dose substance 910 should not occur until the mask is correctly fitted against the face. Correct fitting of the mask 500 against the face is generally necessary to optimise the various functionalities of the mask discussed above, particularly those associated with its protective function (i.e. preventing harmful disturbance from external air currents and/or for controlling the airflow in the region of the eye) and for achieving correct alignment of the propulsion system 908 relative to the eye for ensuring optimal targeting of the dose substance.

The propulsion actuation controller 906 may be configured to trigger operation of the propulsion system 908 automatically when a suitable set of readings from the pressure sensors 902 is received. This arrangement makes the delivery device easier and quicker to use. As soon as the delivery device is in the correct position for delivery, the dose substance is propelled towards the eye and the process is completed.

The device may configured so that the impact of the dose substance onto the eye is recognisable by the user, so that the user is provided with feedback and can be sure that the delivery process has taken place successively.

In the above embodiments, the propulsion system is based on providing a flow of gas over the dose substance exposed in the pocket, to thereby entrain particles of the dose substance towards the eye. However, other arrangements are possible.

For example, the pocket 100 may be attached to, or form part of, a mechanically bi-stable element that can be triggered to transition between a first stable state (i.e. first local energy minimum) and a second stable state (i.e. second local energy minimum). The pocket 100 is arranged such that switching from the first stable state to the second stable state causes rapid movement of the pocket 100 and expulsion of dose substance from the pocket 100 towards the eye.

An example arrangement is shown schematically in FIGS. 10A and 10B. Here, a sectional view of a bi-stable element 1008A/1008B is shown clamped between supports 1000. FIG. 10A shows the element in a first stable state 1008A, which comprises a concave buckle 1009 in a central portion thereof (viewed from the right-hand side in the Figure). FIG. 10B shows the same element in a second stable state 1008B, which is entirely convex. The propulsion system comprises a trigger 1004/1006 consisting of a piston 1006 and a cylinder 1004 and means (not shown) for driving the piston 1006 relative to the cylinder 1004. The bi-stable element can be driven from the first stable state 1008A to the second stable state 1008B by pushing the cylinder 1006 into contact with the buckled central portion 1009 from the left-hand side thereof as shown until it snaps into the second stable state 1008B. The buckled central portion 1009 can act as a pocket for the dose substance or a pocket can be formed separately on or adhered to this region of the element. When the transition from the first stable state 1008A to the second stable state 1008B is triggered, this central portion 1009 accelerates and decelerates rapidly to adopt the second stable state 1008B. During the rapid deceleration phase, powder 1002A in the pocket leaves the pocket and forms a flux 1002B towards the eye as shown in FIG. 10B.

FIGS. 11A and 11B show an alternative arrangement. As in the embodiment of FIGS. 10A and 10B, a flexible member 1100 is supported by clamps 1000. In this embodiment, force is applied to the powder 1002A located on one side of the flexible member 1100 (in a pocket or contained in a region of the flexible member 1100 serving as a pocket) via a force imparting device 1004/1006 on the other side of the flexible member 1100 (in the embodiment shown, the force imparting unit 1004/1006 is based on the same piston/cylinder construction as the trigger of FIGS. 10A and 10B, but other arrangements are possible). The impact between the advancing piston 1006 and the flexible membrane 1100 causes a central portion of the flexible membrane 1100 to accelerate to the right (in the sense of the Figures) and the elastic properties of the flexible member 1100 cause a subsequent rapid deceleration. During this deceleration phase, accelerated powder 1002A leaves the pocket to form a flux 1002B towards the eye as shown in FIG. 11B.

FIG. 12 depicts an alternative means for filtering particles of dose substance according to their mass in order to control the size of particles that reach the eye. Here, the propulsion system comprises a channel 1200 having a portion 1202 downstream of said pocket 100 that is shaped so as to change a direction of flow of the gas as it flows through the portion 1202. The portion 1202 is curved with a constant radius of curvature in the example, but other shapes could also be used. The change of direction of gas as it follows the curve of the channel portion 1202 is effective to change a direction of a flow of dose substance 1204 entrained by the gas as it travels through the portion 1202. However, the dose substance will not generally be able to follow the gas flow completely because the particles of dose substance have greater mass than the particles of gas. The extent to which the particles of dose substance will follow the gas will depend on their mass and surface area and this effect can be used to separate the particles according to contributions from their mass and size. In the example shown, this is achieved by providing an opening 1208 in the curved channel portion 1202 at a position which is too far around the curved portion 1202 for particles that are larger in terms of mass and surface area than a predetermined threshold to flow through the opening 1208. Line 1204A is an example trajectory for such larger particles. Line 1204B shows an example trajectory of particles within a desired size range. These particles leave the channel 1200 via the opening 1208 and are directed towards the eye. Traps may be provided for trapping the particles that are too large to follow trajectories passing through the opening 1208. In the example shown, the opening 1208 is arranged in the side of the channel 1202, but where the aim is only to filter out larger particles, the opening could also be arranged in the end of the channel 1202 (i.e. in a plane perpendicular to the axis of the channel rather than a plane parallel to a portion of the channel wall).

In the above-described embodiments, the various gas sources referred to can be provided in a variety of different forms. For example, the gas sources may be based on means for channeling gas provided manually by a user, for example by manual compression of a plunger in a cylinder or of a bellows system, or the user might blow down a tube or similar. Alternatively, the gas sources may operate by controlled release of gas from a pressurized gas source, such as a compressed gas cylinder. Alternatively, a liquefied-gas source may be used, which has the advantage that the output pressure does not drop with time.

1. A delivery device for delivering a dose substance in powdered form to an eye, comprising:
a pocket for holding dose substance; and
a propulsion system for propelling a dose consisting of a predetermined volume of said dose substance from said pocket towards said eye; wherein
one or both of said pocket and said propulsion system are configured to inhibit propulsion of particles having a size greater than a predetermined threshold to said eye.

2. A delivery device according to claim 1, wherein said propulsion system is configured to propel dose substance from a propulsion area of said pocket, said propulsion area being of a geometry that allows the particles making up said dose to be distributed over said propulsion area in a thin layer, such that the thickness of said layer at any given point is less than 10 percent of the lateral width of said layer, and wherein said delivery device further comprises said thin layer of particles positioned on said propulsion area.

3. (canceled)

4. (canceled)

5. A delivery device according to claim 1, wherein said propulsion system comprises:
a gas source for providing a flow of gas to said pocket that is effective to propel said dose towards said eye;
a channel having a portion downstream of said pocket that is shaped so as to change a direction of flow of said gas as it flows through said portion, said change in direction of gas being effective to change a direction of dose substance entrained by said gas as it travels through said portion; and
a channel opening in said portion of the channel, located so that particles of said dose substance of a predetermined range of sizes will pass through said opening and exit the channel and particles outside of said predetermined range of sizes will not exit the channel via the opening.

6. A delivery device according to claim 1, further comprising:
a charging device for applying an electrostatic charge to particles such that said particles are repelled from each other, wherein said charging device is configured to apply an electrostatic charge to said particles via contact with said pocket.

7. (canceled)

8. A delivery device according to claim 6, further comprising means for controlling the electric field in a region downstream from said pocket so as to modify the cross-sectional shape of the flux of dose substance.

9. A delivery device according to claim 6, wherein said charging device is configured to apply a charge to said particles that causes them to be attracted to said eye, wherein said delivery device comprises an electrode for making electrical contact with the human or animal body to which said eye belongs and for controlling thereby the electric field between the device and said eye in order to ensure that said particles are electrostatically attracted to said eye.

10-14. (canceled)

15. A delivery device for delivering a dose substance in powdered form to an eye, comprising:
a pocket for holding dose substance; and
a propulsion system for propelling a dose consisting of a predetermined volume of said dose substance from said pocket towards said eye; said propulsion system comprising:
a primary gas source for providing a primary flow of gas that flows over or through said pocket and is effective to propel said dose towards said eye; and
a secondary gas source for providing a secondary flow of gas that is separate from said primary flow of gas and which is effective for increasing the proportion of said dose that impinges on said eye.

16. A delivery device according to claim 15, wherein:
said secondary gas source is configured such that said secondary flow of gas interferes with said primary flow of gas in order to reduce the velocity of gas flow near the eye.

17. A delivery device according to claim 15, wherein:
said secondary gas source is configured such that said secondary flow of gas provides a protective buffer that at least partially surrounds said eye before or during delivery of the dose substance.

18. A delivery device according to claim 15, wherein said secondary gas source is configured to direct said secondary flow of gas onto said eye to induce blinking at a point in time prior to delivery of said dose substance that is effective to reduce the probability of blinking during said delivery.

19. A delivery device for delivering a dose substance in powdered form to an eye, comprising:
a pocket for holding dose substance;
a propulsion system for propelling a dose consisting of a predetermined volume of said dose substance from said pocket towards said eye; and
a mask configured in use to enclose a volume delimited by said mask and a portion of a face including said eye and through which volume said dose is to be propelled from said pocket to said eye.

20. (canceled)

21. A delivery device according to claim 19, wherein said mask further comprises a focus target, which is visible to a user when the mask is fitted against said face and which is positioned such that, in use, when dose substance is delivered to said eye while a user is directing his line of sight towards said focus target, subsequent change of said user's line of sight towards a more forward looking direction causes dose substance delivered to said eye to be moved to a portion of said eye in respect of which a residency time is longer.

22. A delivery device according to claim 19, further comprising:
a mask humidity controller for controlling the relative humidity of the air within said mask.

23. A delivery device according to claim 19, wherein said mask is configured to restrain blinking when pressed against said face.

24. A delivery device according to claim 19, further comprising:
a sensor for measuring a contact pressure between a portion of a leading edge of said mask and said face; and
an actuation controller that is configured to allow propulsion of said dose only if a contact pressure measured by said pressure sensor is above a predetermined threshold pressure.

25. A delivery device according to claim 24, wherein said actuation controller is configured automatically to initiate propulsion of said dose when a contact pressure measured by said pressure sensor exceeds said predetermined threshold pressure.

26. A delivery device according to claim 1, comprising:
a pre-dose gas source configured to provide a flow of gas onto said eye prior to propulsion of said dose towards said eye, wherein said pre-dose gas source is configured such that said flow of gas provided by said pre-dose gas source is such as to reduce the surface fluid content of said eye and thereby increase the viscosity of fluid on the surface of said eye.

27. (canceled)

28. A delivery device according to claim 1, further comprising:
a post-dose gas source configured to provide a flow of gas onto said eye after said dose has been propelled towards said eye; and
a humidity source for adding water droplets and/or water vapour to said flow of gas provided by said post-dose gas source.

29-31. (canceled)

32. A delivery device according to claim 1, wherein said propulsion system comprises:
a mechanically bi-stable element which can be triggered so as to undergo a transition between a first stable state and a second stable state, said transition involving rapid movement of a portion of said bi-stable element; and
a trigger for triggering said transition, wherein:
said pocket is located on said portion of said bi-stable element and configured such that, in use, said rapid movement on triggering of said bi-stable element causes said dose to be propelled towards said eye.

33. A delivery device according to claim 1, wherein said propulsion system comprises:
a flexible membrane; and
a force imparting device for applying a force to said flexible membrane from a first side thereof, wherein:
said pocket is located on a second side of said flexible membrane, opposite to said first side, said propulsion system being configured such that said force applied by said force imparting device is such as to cause said dose to be propelled towards said eye.

34-37. (canceled)
